(19) United States
(12) Patent Application Publication
(10) Pub. No.: US 2023/0397813 A1
(43) Pub. Date: Dec. 14, 2023

(54) Health Monitor Device and Method of Use Thereof

(71) Applicants: Yong Chao Zhang, Alameda, CA (US); Bing Zhang, Alameda, CA (US)

(72) Inventors: Yong Chao Zhang, Alameda, CA (US); Bing Zhang, Alameda, CA (US)

(21) Appl. No.: 18/062,759

(22) Filed: Dec. 7, 2022

Related U.S. Application Data
(60) Provisional application No. 63/350,594, filed on Jun. 9, 2022.

(51) Int. Cl. A61B 5/00; A61B 5/0205; A61B 5/11; H02J 7/00

(52) U.S. Cl. CPC A61B 5/002; A61B 5/02055; A61B 5/681; A61B 5/746; A61B 5/4806; A61B 5/1114; H02J 7/0063; A61B 5/02438

(57) ABSTRACT

The present invention relates to an apparatus for independently measuring physiological signals of a user and transmitting same to a wearable device having a display screen for display. The apparatus includes a plurality of sensors including at least a temperature sensor, a blood glucose sensor, an ECG sensor, a SpO2 sensor for measuring physiological signals of the user. The apparatus also includes a battery for providing electric power to the sensors and a processor for processing the signals and wirelessly transmitting same to the wearable device such as a smartwatch for display. The apparatus can be releasably secured to a wearable device such as a smartwatch or wireless blood pressure cuff, thereby extending battery life of the smartwatch by operating independently from the battery of the smartwatch.

### CROSS-REFERENCE TO RELATED APPLICATION

The present application claims priority to, and the benefit of, U.S. Provisional Application No. 63/350,594, which was filed on Jun. 9, 2022, and is incorporated herein by reference in its entirety.

### FIELD OF THE INVENTION

The present invention relates generally to the field of health monitoring devices. More specifically, the present invention relates to a novel health monitor device that can releasably secure and wirelessly couple to a wearable device such as a smartwatch. The device has a battery and a plurality of sensors for independently measuring vitals and other health signals of a user for processing and transmitting to the wearable device for display. The device helps in extending battery life of a smartwatch while still continuously monitoring several aspects of one's health. Accordingly, the present disclosure makes specific reference thereto. Nonetheless, it is to be appreciated that aspects of the present invention are also equally applicable to other like applications, devices, and methods of manufacture.

### BACKGROUND

By way of background, wearable devices are becoming increasingly commonplace to monitor the health of a user by measuring vital signals, track a user's exercise and fitness progress. In certain applications, wearable devices may be configured to measure and monitor signals indicative of electrical activities of the user's heart, such as ECG signals, for detection of certain heart related diseases.

Due to continuous health monitoring by wearable devices such as smartwatches, cuff monitors (i.e., blood pressure (BP), Fitbit, et. al.) and more, power consumption of such devices increases while chances of overheating also increases. Generally, batteries of most smartwatches are designed to last for at least a day, but due to continuous health monitoring, the battery may die in 4-8 hours. Most of the battery power is consumed in monitoring and processing of the health signals. Therefore, individuals are required to repeatedly charge their smartwatches which is not only discomforting, but prevents individuals from continuously wearing their wearable devices. As a result of fast draining of the battery of their wearable devices, individuals generally disable continuous heart rate monitoring, health notifications, stress monitoring, movement monitoring, and more. Disabling such features can be harmful and even fatal for a user as the wearable device may not be able to alert the wearer in case of an emergency. Some smartwatches may not provide accurate tracking for health and vital signs. As a result, individuals desire a device that not only accurately monitors health of a user but also work without using battery of the wearable device such as a smartwatch. More specifically, a device that can work in a manner similar to a smartwatch or similar wearable device without impacting performance of the wearable device is desired by individuals.

Therefore, there exists a long-felt need in the art for a device that keeps track of different vital signs, blood sugar, and more. There is also a long-felt need in the art for a health monitor device that does not operate off the smartwatch' s or similar wearable device's battery power. Additionally, there is a long-felt need in the art for a device that offers a way to maintain extended battery life to a smartwatch. Moreover, there is a long-felt need in the art for a device that can removably attach to a smartwatch or other wearable device enabling the device to monitor health in a similar manner as of the wearable device. Further, there is a long-felt need in the art for a health monitor device that continuously monitors several aspects of one's health throughout the day and night. Furthermore, there is a long-felt need in the art for a device that ensures that the wearable device can remain charged throughout the day while monitoring vitals. Finally, there is a long-felt need in the art for a device that obtains health vital signs without draining a smartwatch battery.

The subject matter disclosed and claimed herein, in one embodiment thereof, comprises a multipurpose health monitor device. The health monitor device is designed to be releasably secured to a wearable device such as a smartwatch for monitoring a plurality of vital signs of a wearer of the smartwatch. The device is positioned such that it maintains contact with the radial artery of a user/wearer and includes a plurality of sensors and detectors for monitoring health parameters of a user. The sensors may include at least a temperature sensor, an ECG sensor, a SPo2 sensor, a blood sugar sensor, and a motion detection sensor. The vital signs measured by said one or more sensors are transmitted wirelessly to the smartwatch to which the device is secured and wirelessly paired using a wireless communication channel. The device offers an accurate method of obtaining health vital signs and includes an integrated battery for performing measurement of vital signs of the wearer. The received vital signs by the smartwatch are displayed on the display screen thereof. The device is further configured to save the battery of the smartwatch as the device does not operate off the watch's battery power.

In this manner, the health monitor device of the present invention accomplishes all of the forgoing objectives and provides users with a health monitor capable of communicating with a smartwatch, enabling users to keep track of different vital signs, blood sugar, and more. The device can be secured to a wearable device for offering a way to maintain extended battery life to a wearable device while still monitoring several aspects such as heartrate, O2 saturation, blood sugar, blood pressure and more of one's health throughout the day and night.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a multipurpose health monitor device. The health monitor device is releasably secured to a wearable smartwatch for monitoring a plurality of vital signs of a wearer of the smartwatch. The device includes a plurality of sensors, including at least a temperature sensor, an ECG sensor, a SPo2 sensor, a blood sugar sensor, and a motion detection sensor wherein the health or vital signs measured by said one or more sensors are transmitted wirelessly to the smartwatch to which the device is secured and wirelessly paired using a wireless communication channel. The device offers an accurate method of obtaining health vital signs and includes an integrated battery for performing measurement of vital signs of the wearer. The received vital signs by the smartwatch are displayed on the display screen thereof wherein the processing of the vital signs is performed by a processor of the health monitor device before transmitting the vital signs information to the smartwatch. The device is further configured to save the battery of the smartwatch as the device does not operate off the watch's battery power and therefore, offers a way to maintain extended battery life to a smartwatch while still monitoring several aspects of one's health throughout the day and night.

In yet another embodiment, an apparatus for measuring vital signs of a user wearing a smartwatch is disclosed. The apparatus comprising an ECG sensor, a blood glucose sensor, a temperature sensor, a SpO2 sensor, a processor, a memory, an accelerometer, wherein the memory configured to store instructions which when executed by the processor become operational with the processor to receive heart rate information from the ECG sensor, blood glucose information from the blood glucose sensor, skin temperature information from the temperature sensor, oxygen saturation information from the SpO2 sensor, and user motion information from the accelerometer, wherein the received sensor information is processed by the processor for making the information compatible to display on a display device of the smartwatch. The apparatus has an integrated battery for providing electric power for functioning of the apparatus, thereby preventing the smartwatch from overheating, and eliminating use of battery of the smartwatch for monitoring vital signs of the user.

In yet another embodiment, a device for measuring and wirelessly displaying vital or physiological signs of a user is disclosed. The device comprising a first portion, including at least one sensor, a second portion having a plurality of adhesive layers, the first portion is releasably attached to the second portion, at least one sensor for measuring at least one vital sign of the user, a processor for receiving and processing the sensor information, a wireless module for transmitting the processed sensor information to a wirelessly coupled smartwatch or blood pressure cuff, a battery for providing the electric power to the sensor and the processor, wherein a display screen of the coupled smartwatch or blood pressure cuff displays the received sensor information.

In yet another embodiment, the sensor may be one of a temperature sensor, an accelerometer, a SpO2 sensor, an ECG sensor, and a blood glucose sensor.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a multipurpose health monitor device of the present invention in accordance with the disclosed architecture;

FIG. 2 illustrates a high-level block diagram showing exemplary electronic and sensor components of the health monitoring device of the present invention in accordance with the disclosed architecture;

FIG. 3 illustrates a perspective view showing the monitoring device being secured to strap of a smartwatch in accordance with the disclosed architecture;

FIG. 4 illustrates a flow diagram depicting a process of pairing the health monitoring device with an external device for displaying the measured vital signals in accordance with the disclosed architecture;

FIG. 5 illustrates a partial perspective view showing the multipurpose health monitor device being used by a user by securing to the strap of a smartwatch in accordance with the disclosed architecture; and

FIG. 6 illustrates a perspective view showing the monitor device monitor being wirelessly connected to a blood pressure cuff in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a device that keeps track of different vital signs, blood sugar, and more. There is also a long-felt need in the art for a health monitor device that does not operate off the smartwatch' s or similar wearable device's battery power. Additionally, there is a long-felt need in the art for a device that offers a way to maintain extended battery life to a smartwatch. Moreover, there is a long-felt need in the art for a device that can removably attach to a smartwatch or other wearable device enabling the device to monitor health in a similar manner as of the wearable device. Further, there is a long-felt need in the art for a health monitor device that continuously monitors several aspects of one's health throughout the day and night. Furthermore, there is a long-felt need in the art for a device that ensures that the wearable device can remain charged throughout the day while monitoring vitals. Finally, there is a long-felt need in the art for a device that obtains health vital signs without draining a smartwatch battery.

The present invention, in one exemplary embodiment, is an apparatus for measuring vital signs of a user wearing a smartwatch or a blood pressure cuff. The apparatus comprising an ECG sensor, a blood glucose sensor, a temperature sensor, a SpO2 sensor, a processor, a memory, an accelerometer, the memory configured to store instructions which when executed by the processor become operational with the processor to receive heart rate information from the ECG sensor, blood glucose information from the blood glucose sensor, skin temperature information from the temperature sensor, oxygen saturation information from the SpO2 sensor and user motion information from the accelerometer, wherein the received sensor information is processed by the processor for making the information compatible to display on a display device of the smartwatch or cuff. The apparatus has an integrated battery for providing electric power for functioning of the apparatus, thereby preventing the smartwatch or cuff from overheating, and eliminating use of battery of the smartwatch for monitoring vital signs of the user.

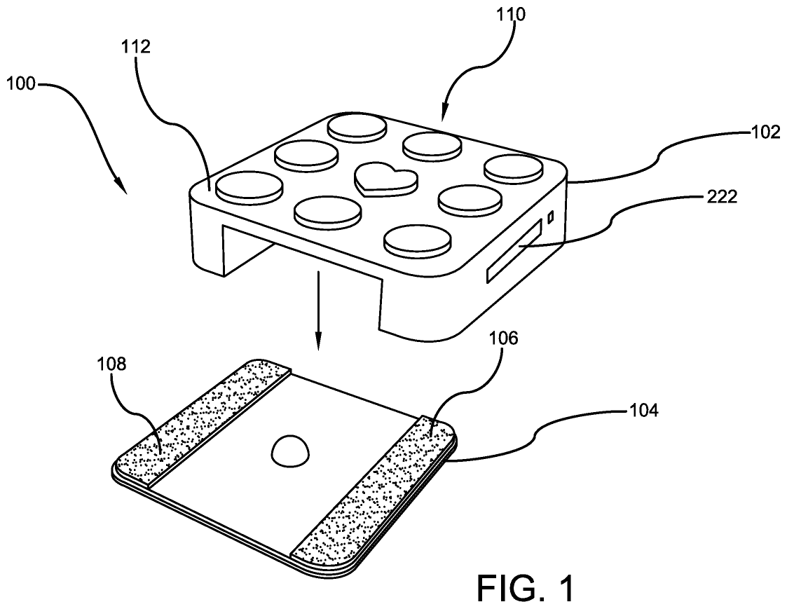

Referring initially to the drawings, FIG. 1 illustrates a perspective view of one potential embodiment of a multipurpose health monitor device of the present invention in accordance with the disclosed architecture. The multipurpose health monitor device 100 of the present embodiment is designed as a compact device capable of wirelessly coupling with a smartwatch worn by a user. The device 100 is configured to monitor different vital signs, blood sugar, and more, and is maintained in a position for contacting the radial artery for accurate pulse and blood pressure monitoring, such that the measured vital signs and other information are wirelessly transmitted to the smartwatch for display and additional processing. As a result, the monitoring device 100 offers a way to maintain extended battery life to a smartwatch while still monitoring health parameters in a non-invasive manner throughout the day and night.

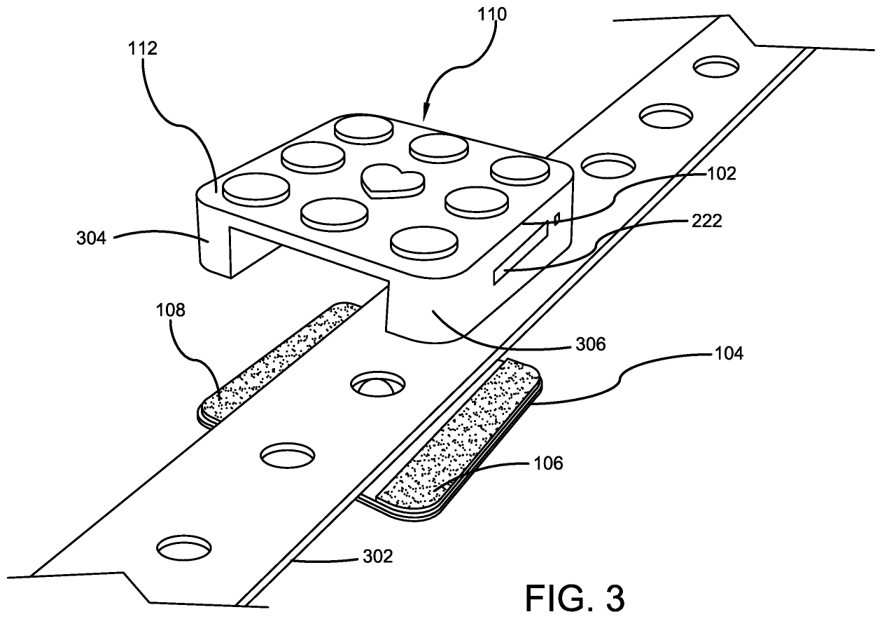

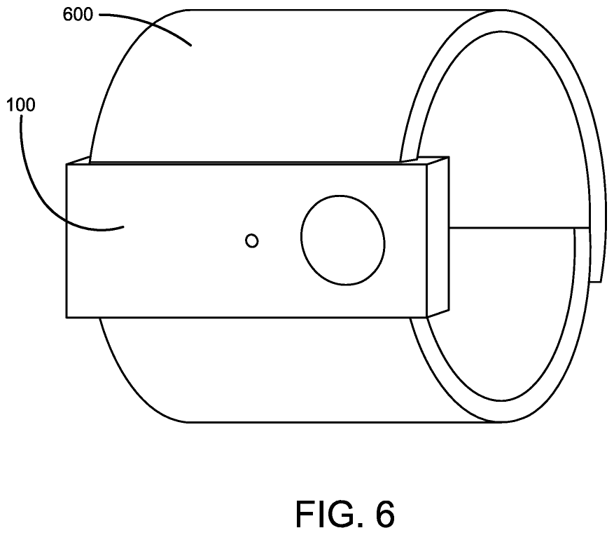

More specifically, the device 100 has a first portion 102 and a second portion 104 such that the first portion 102 and the second portion 104 are releasably attached to each other. The first portion 102 includes a plurality of electronic modules and sensors for monitoring vital signs and other biophysical parameters of a user using the device 100 and the second portion 104 is used for securing the device 100 to a strap of a smartwatch, for example. The first portion 102 is releasably attached to adhesive layers 106, 108 disposed on the second portion 104 such that the device 100 can easily secure to strap of a smartwatch as illustrated in FIG. 3 or to a wireless blood pressure cuff as illustrated in FIG. 6.

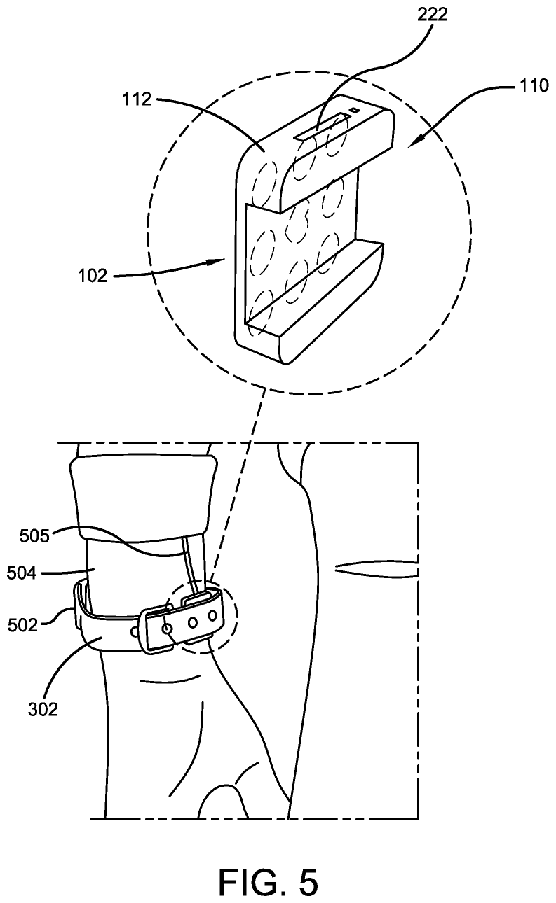

In use, the first portion 102 touches the skin and maintains contact with a radial artery of a user for measuring vital signs and transmitting to a coupled smartwatch. This obviates use of the battery of the smartwatch and offers a more accurate method of obtaining health vital signs of the user as illustrated in FIG. 5. The plurality of the sensors 110 are positioned on the top surface 112 of the first portion 102 enabling the sensors 110 to touch the skin of the wearer and enabling easy measurement of the vital and other biophysical signs of the user.

When the device 100 is required to be recharged or not used, the second portion 104 is detached from the first portion 102 enabling the device 100 to be removed from smartwatch or cuff. The device 100 when formed by attaching the portions 102, 104, forms a cavity between the portions 102, 104 enabling the device 100 to secure to a smartwatch strap, cuff, and any other similar device.

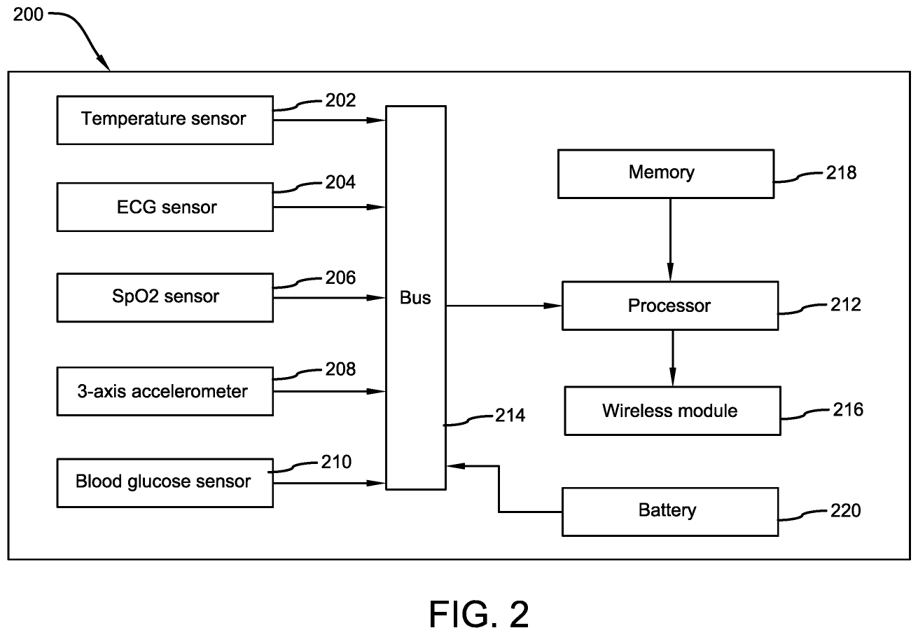

FIG. 2 illustrates a high-level block diagram showing exemplary electronic and sensor components of the health monitoring device 100 of the present invention in accordance with the disclosed architecture. The block diagram 200 shows the exemplary sensors and processing modules included in the device 100 that provide the functionality of measuring and transmitting vital signs information of a user. A temperature sensor 202 is used for measuring skin temperature of a user wearing the device 100 and touching the skin of the user. The temperature sensor 202 can be in the form of thermocouple or thermistor mounted in the first portion 102 of the device 100. The temperature readings measured by the sensor 202 are transmitted to a coupled smartwatch via a wireless connection for making decisions such as providing an indication of an alarm condition (e.g., a high body temperature condition or a loss of thermal contact between the sensor 202 and the skin of the user).

An electrocardiogram (ECG) sensor 204 is configured to create an ECG/EKG graph for the user and to help identify atrial fibrillation (Afib). Preferably, the sensor 204 measures the bio-potential generated by electrical signals that control the expansion and contraction of heart chambers. In some embodiments, a PPG (photoplethysmography) sensor is used in place of the ECG sensor and uses a light-based technology to sense the rate of blood flow as controlled by the heart's pumping action. The PPG sensor generally uses green-light PPG, preferably to measure heart rate while the user is in motion.

A SpO2 sensor 206 is used for measuring blood oxygen levels via red and infrared light sensors. The sensor 206 works on the principle of reflectance oximetry in which SPO2 is measured through the light that reflects from the blood under the skin. A 3-axis accelerometer 208 is included in the device 100 and is used for detecting wrist movement detection of the user enabling the device 100 to detect movement of the user using the device 100 to track sleep and sleep patterns. The accelerometer data along with the heart rate data is used for measuring estimation of night sleep and inactiveness/activeness of a user.

A blood glucose sensor 210 is configured to monitor glucose level continuously throughout the day and night in a non-invasive manner. The sensor 210 includes a plurality of micro points and biosensors that measure glucose painlessly through the skin. A processor 212 is configured to send and receive data from one or more sensors of the device 100 along an internal bus 214, which can be in the form of one or more wire traces on an integrated chip. It should be noted that any conventional communication means can be used for communication between the sensors and the processor 212. The processor 212 may include a general-purpose processor or a specific-purpose processor and may be may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable device or a combination of devices that can perform calculations or other manipulations of information.

A wireless module 216 is configured to establish a wireless, preferably Bluetooth channel for coupling the device 100 to a smartwatch thereby enabling the processor 212 of the device 100 to wirelessly transmit the vital and other biosignal information for display and further processing. It should be appreciated that the processor 212 is configured to process the sensor signals in a format compatible with the paired smartwatch, thereby enabling the smartwatch to display the information on the display device thereof. In certain embodiments, the transmitter 216 includes one or more of an RF signal generator (e.g., an oscillator), a modulator (a mixer), and a transmitting antenna.

An internal memory 218 is configured to store data and/or instructions for software programs for processing the sensor signals and information. The instructions stored in the memory 218 enables the processor to perform communication signals through the wireless module 216 and for generating alerts based on the detected values by one or more sensors. The memory 218 can be volatile or non-volatile memory, including, but not limited to, a Random Access Memory (RAM), a flash memory, a Read Only Memory (ROM), a Programmable Read-Only Memory (PROM), an Erasable PROM (EPROM), registers, a hard disk, a removable disk, or any other suitable storage device. Memory 218 may also store established thresholds for each sensor of the device 100, thereby enabling the processor 212 to generate safety thresholds.

A button battery 220 provides electric power for working of the device 100 and a charging port 222 (shown in FIG. 1) is provided for recharging the battery 220. The battery 220 enables the device 100 to work independently of the smartwatch or any other external device and therefore, the device 100 is able to free workload from the smartwatch and extend battery life to a smartwatch while still monitoring several aspects of one's health throughout the day and night. The device 100, unlike a patch, is not required to stick to the skin or body of the user, but can be secured to a strap of the smartwatch or cuff, thereby providing ease and comfort to a user using the device 100.

FIG. 3 illustrates a perspective view showing monitoring device 100 being secured to a strap of a smartwatch in accordance with the disclosed architecture. For releasably securing the device 100 to a strap 302 of a smartwatch, the second portion 104 is placed below the strap 302 such that the adhesive bands or layers 106, 108 face the strap 302. Then, the first portion 102 is positioned on the top of the strap 302 such that the side edges 304, 306 of the first portion 102 are positioned on the adhesive bands 106, 108 to attach to the second portion 104 such that a portion of the strap 302 is overlapped by the device 100.

The top surface 112 of the first portion 102 is positioned to touch the skin and, in one exemplary embodiment, to maintain contact with the radial artery of the user wearing the strap 302 such that the plurality of electrodes/sensors/detectors touch the skin and maintains contact with the radial artery of the user to measure various vital and other bio signals and wirelessly transmit same to the paired smartwatch. The radial artery includes the radial pulse which can be felt and detected on the wrist, just under the thumb.

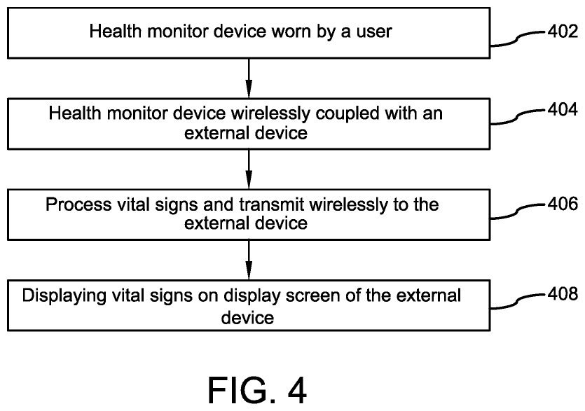

FIG. 4 illustrates a flow diagram depicting a process of pairing the health monitoring device with an external device for displaying the measured vital signals in accordance with the disclosed architecture. As will be apparent to one of ordinary skill in the art, there are numerous variations of the principles disclosed in this specific example and in the examples that follow. Steps may be performed in a different order, some steps omitted, other steps added, and the logic flow may be varied without departing from the scope of the subject disclosure.

Initially, the health monitor device 100 is secured to the external device such that the device touches the skin of a subject user when the external device is won by the user (Step 402). In various embodiments of the invention, the external device can be a smartwatch or wireless blood pressure monitoring cuff.

Thereafter, the health monitor device is wirelessly coupled or paired with the external device (Step 404) enabling the device to transmit the measured health activity to the external device at predetermined intervals or during the detection of any emergency situation (Step 406). The emergency situation can be deduced by the device 100 based on comparison of the measured sensor values with one or more predetermined thresholds stored in the internal memory of the device 100. Finally, the information received by the external device can be displayed on the displayed device thereof or on a smartphone paired with the external device (Step 408).

FIG. 5 illustrates a partial perspective view showing the multipurpose health monitor device being used by a user by securing to the strap of a smartwatch in accordance with the disclosed architecture. As illustrated, the device 100 is secured to the strap 302 in a manner described in FIG. 3 and the smartwatch 502 is worn by a user around the wrist 504 such that the top surface 112 of the first portion 102 touches the skin and the wrist radial artery 505 of the user.

The sensors disposed in the first portion 102 are configured to continuously monitor various health parameters of the user as described in FIG. 2.

The smartwatch 502 receives the sensor data in digital form from the device 100 and is displayed on the display device of the smartwatch 502. The device 100 connects wirelessly with the smartwatch 502 rather than operating off the watch's battery power ensuring the watch 502 can remain charged throughout the day while monitoring vitals of the user. The device 100 prevents the smartwatch 502 from overheating since the independent device 100 has its own battery source/power as described in FIG. 2.

FIG. 6 illustrates a perspective view showing the monitor device monitor being wirelessly connected to a blood pressure cuff in accordance with the disclosed architecture. The device 100 is releasably attached to the blood pressure cuff 600 enabling the device 100 to touch the skin of a wearer of the cuff 600 such that sensors of the device 100 continuously monitors the vitals of the wearer of the cuff 600 and the measured vitals are displayed on a display screen of the cuff device 600.

It should be noted that any additional sensors to enhance the utility of the device can be included in the device 100 and the communication of the additional sensor information is transmitted to the smartwatch or any other external device to which the device 100 is connected. The battery and sensors may also be comprised of a soft, flexible material.

The external device to which the health monitoring device 100 can transmit vital signs data can be implemented by any configuration of one or more computers, such as a microcomputer, a super computer, a general-purpose computer, a special-purpose/dedicated computer, a remote server computer, a personal computer, a laptop computer, a tablet computer, a cell phone, a personal data assistant (PDA), a wearable computing device, e.g., a smart watch, or a computing service provided by a computing service provider, e.g., a website, or a cloud service provider. In some implementations, the external device can be a smart phone device that can be used to display and analyze vital signs signals.

Vital signs information, data, and signals can be represented using a variety of different technologies and techniques. For example, any data, instructions, commands, information, signals, bits, symbols, and chips referenced herein can be represented by voltages, currents, electromagnetic waves, magnetic fields or particles, optical fields or particles, other items, or a combination of the foregoing.

Implementations, or portions of implementations, of the above disclosure can take the form of a computer program product accessible from, for example, a computer-usable or computer-readable medium. A computer-usable or computer-readable medium can be any device that can, for example, tangibly contain, store, communicate, or transport a program or data structure for use by or in connection with any processor. The medium can be, for example, an electronic, magnetic, optical, electromagnetic, or a semiconductor device.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “health monitoring device”, “device”, “multipurpose health monitor device”, and “monitoring device” are interchangeable and refer to the multipurpose health monitor device 100 of the present invention.

Notwithstanding the forgoing, the multipurpose health monitor device 100 of the present invention can be of any suitable size and configuration as is known in the art without affecting the overall concept of the invention, provided that it accomplishes the above stated objectives. One of ordinary skill in the art will appreciate that the multipurpose health monitor device 100 as shown in the FIGS. are for illustrative purposes only, and that many other sizes and shapes of the multipurpose health monitor device 100 are well within the scope of the present disclosure. Although the dimensions of the multipurpose health monitor device 100 are important design parameters for user convenience, the multipurpose health monitor device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A multipurpose health monitor device for wirelessly coupling with a smartwatch, the multipurpose health monitor device comprising:
a health monitor device worn by a user having a plurality of sensors for monitoring vital signs selected from a group consisting of a pulse, a blood pressure and a temperature;
a battery source for independently powering said health monitor device;
wherein said vital signs are wirelessly transmitted to a smartwatch;
wherein said health monitor device positioned around a strap of the smartwatch to maintain contact with a radial artery of the user;
wherein said health monitor device having a first portion and a second portion wherein said first portion and said second portion are releasably attached to one another;
wherein said first portion having a plurality of electronic modules comprising said plurality of sensors for monitoring said vital signs; and
further wherein said first portion and said second portion having an attachment mechanism for securing said health monitor device to the strap of the smartwatch.

2. The multipurpose health monitor device of claim 1, wherein said attachment mechanism having adhesive layers disposed on said second portion for releasable attachment to said first portion and for securing said strap of the smartwatch between said first portion and said second portion.

3. The multipurpose health monitor device of claim 2, wherein the smartwatch is a wireless blood pressure cuff.

4. The multipurpose health monitor device of claim 2, wherein said vital signs having biophysical parameters.

5. The multipurpose health monitor device of claim 4, wherein said first portion touches the skin of the user including the radial artery of the user for measuring said vital signs and transmitting said vital signs to the smartwatch.

6. The multipurpose health monitor device of claim 5, wherein said plurality of sensors positioned on a top surface of said first portion when in a mounted orientation for touching the skin of the user.

7. The multipurpose health monitor device of claim 6, wherein said first portion and said second portion when attached having a cavity between said first portion and said second portion to secure said strap of the smartwatch therein.

8. The multipurpose health monitor device of claim 7, wherein said temperature sensor measures skin temperature of the user.

9. The multipurpose health monitor device of claim 8, wherein said temperature sensor is selected from a group consisting of a thermocouple and a thermistor mounted in said first portion of said health monitor device.

10. The multipurpose health monitor device of claim 9, wherein monitored said vital signs are compared to a preset alarm condition.

11. The multipurpose health monitor device of claim 10, wherein said health monitor device providing an indication of said preset alarm condition when said monitored said vital signs are above said preset alarm condition.

12. The multipurpose health monitor device of claim 11, wherein said plurality of sensors for monitoring vital signs further selected from a group consisting of an electrocardiogram (ECG) sensor, a photoplethysmography (PPG) sensor, and a SpO2 sensor.

13. The multipurpose health monitor device of claim 12, wherein said plurality of sensors further comprising a 3-axis accelerometer for detecting wrist movement of the user for tracking sleep patterns.

14. The multipurpose health monitor device of claim 13, wherein said plurality of sensors further comprising a plurality of micro points having a blood glucose sensor for monitoring a glucose level of the user.

15. The multipurpose health monitor device of claim 14 further comprising communication mechanism for communicating between said plurality of sensors and a processor, wherein said processor is selected from a group consisting of a general purpose processor, a specific-purpose processor, a microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, and a gated logic.

16. A multipurpose health monitor device for wirelessly coupling with a smartwatch, the multipurpose health monitor device comprising:
a health monitor device worn by a user having a plurality of sensors for monitoring vital signs selected from a group consisting of a pulse, a blood pressure, and a temperature;
a battery source for independently powering said health monitor device;
wherein said vital signs are wirelessly transmitted to a smartwatch;
wherein said health monitor device is positioned around a strap of the smartwatch to maintain contact with a radial artery of the user;
wherein said health monitor device having a first portion and a second portion wherein said first portion and said second portion are releasably attached to one another;
wherein said first portion having a plurality of electronic modules comprising said plurality of sensors for monitoring said vital signs;
wherein said first portion and said second portion having an attachment mechanism for securing said health monitor device to the strap of the smartwatch; and
a wireless module configured to establish a wireless channel for coupling said health monitor device to the smartwatch enabling a processor of said health monitor device to wirelessly transmit said vital signs.

17. The multipurpose health monitor device of claim 16, wherein said temperature sensor measures skin temperature of the user.

18. The multipurpose health monitor device of claim 17, wherein said temperature sensor is selected from a group consisting of a thermocouple and a thermistor mounted in said first portion of said health monitor device.

19. A multipurpose health monitor device for wirelessly coupling with a smartwatch, the multipurpose health monitor device comprising:
a health monitor device worn by a user having a plurality of sensors for monitoring vital signs selected from a group consisting of a pulse, a blood pressure, and a temperature;
a battery source for independently powering said health monitor device;
wherein said vital signs are wirelessly transmitted to a smartwatch;
wherein said health monitor device positioned around a strap of the smartwatch to maintain contact with a radial artery of the user;
wherein said health monitor device having a first portion and a second portion wherein said first portion and said second portion are releasably attached to one another;
wherein said first portion having a plurality of electronic modules comprising said plurality of sensors for monitoring said vital signs;
wherein said first portion and said second portion having an attachment mechanism for securing said health monitor device to the strap of the smartwatch;
a wireless module configured to establish a wireless channel for coupling said health monitor device to the smartwatch enabling said processor of said health monitor device to wireles sly transmit said vital signs; and
further wherein monitored said vital signs are compared to a preset alarm condition.

20. The multipurpose health monitor device of claim 19, wherein said health monitor device providing an indication of said preset alarm condition when said monitored said vital signs are above said preset alarm condition.
